(19) United States
(12) Patent Application Publication
(10) Pub. No.: US 2003/0229029 A1
(43) Pub. Date: Dec. 11, 2003

(54) Cardiac glycosides for treating muscle pain and spasm

(76) Inventors: Charles Laudadio, Chadds Ford, PA (US); Matthew Davis, Media, PA (US)

Correspondence Address: Connolly Bove Lodge & Hutz LLP, P.O. Box 2207, Wilmington, DE, 19899-2207, US

(21) Appl. No.: 10/454,065

(22) Filed: Jun. 4, 2003

Related U.S. Application Data
(60) Provisional application No. 60/386,261, filed on Jun. 6, 2002.

(51) Int. Cl. A61K 31/704

(52) U.S. Cl. 514/26

(57) ABSTRACT

This invention provides methods of treating muscle spasm and/or pain by treatment with a cardiac glycoside or aglycone derivative. These methods are useful in treatment of conditions associated with muscle pain.

### CROSS-REFERENCE TO RELATED APPLICATION

This application is based upon provisional application Serial No. 60/386,261, filed Jun. 6, 2002.

### TECHNICAL FIELD

The present invention relates generally to use of administering a cardiac glycoside (topically, orally, parenterally or anally) for the relief of striated muscle pain.

### BACKGROUND OF THE INVENTION

Foxglove is the common name for plants of the Digitalis species, primarily represented by common foxglove, Digitalis purpurea L., and Grecian foxglove, Digitalis lanata J. F. Ehrh. Digitalis is poisonous, and symptoms include vomiting, headache, irregular heartbeat, and convulsions. Overdoses can be fatal. Cardiac glycosides such as digitalis have been used extensively to treat heart failure. The assumed mechanism of action is inhibition of Na⁺, K⁺-ATPase resulting in increased intracellular sodium and subsequent intracellular calcium leading to enhanced muscle contraction in cardiac tissue. This enhanced muscle contraction is thought to include increasing the contractility of the heart. Various cardiac glycosides may have cardiotonic or cardiotoxic effects.

There are many natural, secondary, and other derived compounds from Digitalis species. The important cardenolides or cardiac glycosides include digitoxin, digoxin, gitoxin, from the lanatosides A, B, and C, and from purpurea glycoside A and B. The important saponins include digitonin, tigonin and gitonin. Additional medicinally useful steroids and cortical hormones can be made from the plant steroids. Cardiac glycosides are divided into two main types: Bufadienolides which are C24 steroids and Cardenolides (most prevalent) which are C23 steroids. They have a characteristic 5- or 6-membered lactone ring. They are called cardiac glycosides because they modify heart action. Cardenolides inhibit the Na⁺-K⁺-ATPase pump in mammals.

This group of compounds is found in a large number of families, many of which are unrelated. There are about 400 known cardenolides. A number of toads and frogs make cardiac active compounds that are steroidal, but not glycosidic, in nature.

Cardenolides are derived from steroidal precursors, probably cholesterol, via the intermediacy of pregnenolone or progesterone intermediates. The exact pathway does not seem to have been established with certainty. Many questions about the probable acetate/malonate origin of the five or six membered lactone ring.

Most members of the family Asclepiadaceae contain cardiac glycosides. Several plants with cardiac glycosides or cardenolides are used medicinally. Among these are Digitalis species (Scrophulariaceae) and ouabain (Strophanthus species) (Apocynaceae). These plants are often used to treat heart problems.

Chronic and recurrent muscle pain is the second most commonly diagnosed medical condition after upper respiratory illness. Ten to 20 percent of the American population, over 44 million persons 18 years of age and older, are affected per year. Patients with these conditions make 70 million visits to physicians and 425 million visits to chiropractors and other alternative health care providers each year at an annual cost of $47 billion. It is the single largest diagnostic group in pain clinics, accounting for 85% of pain clinic populations.

Patients with muscle pain carry many different diagnostic labels including muscle strain, whiplash, repetitive overuse syndrome, fibromyalgia, myofascial trigger point pain syndrome, tension headache, and low back syndrome.

A cost effective manner of treating these types of painful myofascial conditions would be very beneficial to healthcare.

It is hypothesized, that the Na⁺-K⁺-ATPase pump may play a role in maintaining myofascial pain by setting up a vicious cycle of spasm and pain of the peripheral striated muscles. It is postulated that the ability to block this cycle of spasm can be accomplished with the administration of a cardiac glycoside which will interfere with the Na⁺-K⁺-ATPase pump, restore normal intracellular electrolytes and relieve muscle pain.

### SUMMARY OF THE INVENTION

Seeds of the plant family APOCYNACEAE contain three glucosids, namely: non-basic, amorphous, poisonous oleandrin, nerein (nerlin), considered to be identical with digitalein, and crystallizable nerianthin, free from nitrogen.

One species of the apocynaceae is Thevetia Thevetioides. It also goes by the name of Thevetia yccotli, De Candolle (Cerbera thevetioides). The tree is known in the Mexican Cordilleras as the joyote. It inhabits the damp, hot sections of the mountains. The fruit is applied to hemorrhoids. The seeds, which are known as joyote seeds, are very acrid and poisonous. A. Herrera (Amer. Jour. Pharm, 1877, p. 145) obtained, by pressure, 40 per cent, of a fixed oil, and a crystallizable, acrid glucosid which he called thevetosin. Merck (1894) isolated another glucosid which he named cerberid. It is a cardiac poison and felt to be fatal if taken internally.

However, the systemic effects of these glycosides are concentration dependent so that at therapeutic levels, cardiac glycosides like digitalis can assist failing hearts but in high doses can be fatal.

Using an extraction in isopropyl alcohol of Thevetia Thevetioides seeds, the resulting liquid attains lethal levels of cardiac glycosides if taken internally, however, when applied topically to painful muscles, results in immediate relief of pain, presumably by amelioration of the muscle spasm. Since this is applied topically, systemic blood levels of cardiac glycosides remain very low and do not cause side effects.

Over a period of 12 years, over 3 dozen patients have been treated with this topical preparation with dramatic clinical benefit (relief of muscle pain and spasm) and no side effects. While the precise mechanism of action has not been determined, the know effect of glycosides on the Na-K pump suggests the biochemical mechanism is probably inhibition of this pump at the peripheral receptor sites of striated peripheral muscle.

Thus, the basis of this invention is the use of glycosides for the treatment of muscle pain and spasm.

### DETAILED DESCRIPTION OF THE INVENTION

“Cardiac glycoside” refers to a group of compounds which are structurally related. Structurally, these compounds are derived from the cyclopentanoperhydro-phenanthrene nucleus characteristic of steroid compounds, have a five-membered unsaturated lactone ring of a six-membered doubly unsaturated lactone ring at C17 of ring D, a hydroxyl group at C3 in ring A for joining by an ether linkage to one or more sugar residues, and a hydroxy group at C14. The aglycone derivatives of cardiac glycosides have a similar structure, but lack the carbohydrates characteristic of the cardiac glycosides. These aglycone derivatives are also useful in the present invention. Representatives of this group are found in a number of botanical sources, as well as in mammals. (See, A survey of Cardiac Glycosides and Genins, University of South Carolina Press, 1961.) The cardiac glycosides include ouabain-like/digoxin-like compounds that have been isolated from mannals (see, U.S. Pat. No. 4,780,314). Other sources of cardiac glycosides include seeds of one or more of the genera of Thevetia (peruviana, neriufolia, thevetiodes, ahouia etc.).

Cardiac glycosides and aglyconesand together, cardiac glycosides and aglycone derivatives are classified as cardenolides. Cerberoside is a cardiotonic compound found in thevetia. Chemicals With Digitalic Activity include:

Cactine

Cassaidine

Cerberin

Cheirotoxin

Erythrophleguine

Hellebrin

K-Strophanthoside

Lanatoside-C

Peruvoside

Thevetin

Cardiac glycosides useful in the present invention include, but are not limited to, lanatoside A, desacretyllanatoside A, actyl digitoxin, digitoxin, lanatoside C, desacetyllanatoside C, digoxin, strophanthoside K-strophanthin, ouabain, scillaren A, proscillaridin A, uzarin, digitoxose, gitoxin, strophanthidine-3 beta-digitoxoside, strophanthidin .alpha.-L-rhamnopyranoside, strophanthidol, oleandrin, acovenoside A, strophanthidine digilanobioside, strophanthidin-D-cymaroside, digitoxigenin-L-rhamnoside digitoxigenin theretoside, and the like. Aglycones include, but are not limited to, strophanthidin, digitoxigenin, uzarigenin, digoxigenin, digoxigenin 3,12-diacetate, gitoxigenin, gitoxigenin 3-acetate, gitoxigenin 3,16-diacetate, 16-acetyl gitoxigenin, acetyl strophanthidin, ouabagenin. 3-epidigoxigenin, and the like. Preferably the cardiac glycoside is ouabain, digoxin, or digitoxin. In a preferred practice, the cardiac glycoside is ouabain, and the aglycone derivative is strophanthidin. In a more preferred practice the cardiac glycoside is liquid digoxin.

Cardiac glycosides and aglycones may be purified from organisms, such as plants, or from human serum or urine. (see, for example, references in Merck Index, Tenth Edition; PCT application WO91/17176; U.S. Pat. No. 4,780,314; Kelly et al., Kidney Int'l 30:723-729, 1986). The compounds may also be purchased commercially (e.g., Sigma Chemical Co., St. Louis, Mo.; Calbiochem San Diego, Calif).

Administration

As described above, cardiac glycosides are a useful method for treating muscle spasm and pain. Treatment means that symptoms may be lessened or the progression of the disease or conditions halted or delayed. Cells to be treated are contacted with a cardiac glycoside or aglycone derivative of a cardiac glycoside at a therapeutically effective dosage. Contacting may be effected by incubation of cells ex vivo or in vivo, such as by topical treatment, delivery by specific carrier or by vascular supply.

The main mode of administration is topical but cardiac glycosides for the treatment of muscle spasm and pain may also be formulated into pharmaceutical compositions suitable for local, intravenous and systemic application. Time release and topical patch formulations are also possible. Effective concentrations of one or more of the conjugates are mixed with a suitable pharmaceutical carrier or vehicle. The concentrations or amounts of the conjugates that are effective requires delivery of an amount, upon administration, that ameliorates the symptoms or treats the disease. Typically, the compositions are formulated for single dosage administration. Therapeutically effective concentrations and amounts may be determined empirically by testing the conjugates in known in vitro and in vivo systems, such as those described herein; dosages for humans or other animals may then be extrapolated therefrom.

Other diseases associated with muscle spasm and pain that may be treated with cardiac glycosides include but are not limited to: Complex Regional Pain Syndromes (I & II), Fibromyalgia, Spastic Torticollis, Low Back Pain, General Myofascial Pain, Neuropathic Pain, Muscle Spasm and Pain secondary to pregnancy, Amyotrophic lateral sclerosis, Cerebral palsy, Cramps, stroke, multiple sclerosis, cerebral palsy, neurodegenerative diseases, trauma, spinal cord injury, and nervous system poisons such as strychnine, tetanus, and certain insecticides. Nerve damage may lead to a prolonged or permanent muscle shortening called contracture. However, most muscle spasms are not caused by disease, but more commonly by physical activity or stress.

Relaxation of a muscle actually requires energy to be expended. The energy is used to recapture calcium and to unlink the actin and myosin. This causes the muscles fibers to lengthen because the unlinked chains slide back to their resting positions. Normally, sensations of pain and fatigue signal that it is time to slow down or stop. Resting allows the muscles to restore their supplies of energy. Ignoring or overriding those warning signals can lead to such severe energy depletion that the muscle cannot be relaxed, causing a cramp. The lack of blood flow deprives the muscles of their source of energizing oxygen and nutrients and removal of fatigue causing waste. Rigor mortis, the stiffness of a corpse within the first 24 hours after death, is also due to this phenomenon.

Anemia adversely effects blood flow to the muscles and can cause cramping and spasms.

Dehydration and salt depletion. This may be brought on by protracted vomiting or diarrhea, or by copious sweating during prolonged exercise, especially in high temperatures. Loss of fluids and salts—especially sodium, potassium, magnesium, and calcium—can disrupt ion balances in both muscle and nerves. This can prevent them from responding and recovering normally, and can lead to a cramp.

Metabolic disorders that affect the energy supply in muscle. These are inherited diseases in which particular muscle enzymes are deficient. They include deficiencies of myophosphorylase (McArdle's disease), phosphorylase b kinase, phosphofructokinase, phosphoglycerate kinase, and lactate dehydrogenase. Myotonia. This causes stiffness due to delayed relaxation of the muscle, but does not cause the spontaneous contraction usually associated with cramps. However, many patients with myotonia do experience cramping from exercise. Symptoms of myotonia are often worse in the cold. Myotonias include myotonic dystrophy, myotonia congenita, paramyotonia congenita, and neuromyotonia.

Vascular disease, such as arteriosclerosis, Reynaud's disease, diabetic vasculopathy, decreases blood flow to muscles, which can cause cramping.

Exposure to cold can also decrease blood flow, resulting in cramping and muscle spasms.

Fasciculations may be due to fatigue, cold, medications, metabolic disorders, nerve damage, or neurodegenerative disease, including amyotrophic lateral sclerosis. Most people experience brief, mild fasciculations from time to time, usually in the calves.

The pain of a muscle cramp is intense, localized, and often debilitating. Coming on quickly, it may last for minutes and fade gradually. Contractures develop more slowly, over days or weeks, and may be permanent if untreated. Fasciculations may occur at rest or after muscle contraction, and may last several minutes. (Hong Kong—The Hong Kong Practitioner, July 2000, p. 345.) Chronic pain is devastating and demoralizing and causes numerous adverse effects, including insomnia, anxiety, impaired concentration and depression.

The three main types of chronic pain are nociceptive (visceral and somatic), neuropathic and muscle spasm Visceral pain originates in hollow organs and frequently presents as colic. Somatic pain originates in tissues such as muscle, bone or skin. Neuropathic pain, which originates in the central or peripheral nervous system, is under-recognized as a cause of chronic pain. Muscle spasm may present as tension headache or shoulder girdle muscle pain and is often related to anxiety and stress. Spinal column pathologies can cause muscle spasm in the paravertebral muscles. Taking a detailed history may be sufficient to identify the source and extent of pain, especially if the physician uses a systematic pain questionnaire. Numerical ratio scales, visual analog scales and verbal descriptor scales have been developed to measure the severity of pain.

Low back pain is a common musculoskeletal symptom that may be either acute or chronic. It may be caused by a variety of diseases and disorders that affect the lumbar spine. Low back pain is often accompanied by sciatica, which is pain that involves the sciatic nerve and is felt in the lower back, the buttocks, and the backs of the thighs.

Pharmaceutical carriers or vehicles suitable for administration of the conjugates provided herein include any such carriers known to those skilled in the art to be suitable for the particular mode of administration. In addition, the inhibitor may be formulated as the sole pharmaceutically active ingredient in the composition or may be combined with other active ingredients.

The compositions of the present invention may be prepared for administration by a variety of different routes. Local administration of the cardiac glycosides or aglycone derivatives is preferred. The inhibitor may be mixed with suitable excipients, such as salts, buffers, stabilizers, and the like. If applied topically, such as to the skin and mucous membranes, the inhibitor may be in the form of gels, creams, and lotions. Such solutions, particularly those intended for ophthalmic use, may be formulated as 0.01%-10% isotonic solutions, pH about 5-7, with appropriate salts.

Solutions or suspensions used for parenteral, intradermal, subcutaneous, or topical application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbin acid and sodium bisulfite; chelating agents, such as ethylenediaminetetraacetic acid (EDTA); buffers, such as acetates, citrates and phosphates; and agents for the adjustment of toxicity such as sodium chloride or dextrose. Parental preparations can be enclosed in ampules, disposable syringes or multiple dose vials made of glass, plastic or other suitable material.

If administered intravenously, suitable carriers include physiological saline or phosphate buffered saline (PBS), and solutions containing thickening and solubilizing agents, such as glucose, polyethylene glycol, and polypropylene glycol and mixtures thereof Liposomal suspensions may also be suitable as pharmaceutically acceptable carriers. There may be prepared according to methods known to those skilled in the art.

The inhibitor may be prepared with carriers that protect it against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydides, polyglycoli acid, polyorthoesters, polylactic acid and others. For example, the compositions may be applied during surgery using a sponge, such as a commercially available surgical.

The inhibitors can be administered by any appropriate route, for example, orally, parenterally, intravenously, intradermally, subcutaneously, or topically, in liquid, semi-liquid or solid form and are formulated in a manner suitable for each route of administration. Preferred modes of administration depend upon the indication treated. Dermatological and ophthalmologic indications will typically be treated locally; whereas, tumors and restenosis will typically be treated by systemic, intradermal or intramuscular modes of administration.

The inhibitor is included in the pharmaceutically acceptable carrier in an amount sufficient to exert a therapeutically useful effect in the absence of undesirable side effects. It is understood that number and degree of side effects depends upon the condition for which the conjugates are administered. For example, certain toxic and undesirable side effects are tolerated when treating life-threatening illnesses, such as tumors, that would not be tolerated when treating disorders of lesser consequence. The concentration of conjugate in the composition will depend on absorption, inactivation and excretion rates thereof, the dosage schedule, and amount administered as well as other factors known to those of skill in the art.

The inhibitor may be administered one time, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Another aspect of the invention is a composition of matter comprising cardiac glycosides for the topical treatment of pain, muscle pain or muscle spasms. Such composition of matter comprises the topical application (such as aqueous acids, aromatic waters, balms, cataplasms [poultices], collodions, creams, dressings, elixirs, emulsions, extracts, fluid extracts, foams, gels, glycerins, honeys, irrigation solutions, jellies, juices, liniments, liposomes, lotions, magmas, microemulsion, milks, mixtures, mucilages, oils, ointments [such as oleaginous, anhydrous, water in oil, oil in water and water soluble bases], oleovitamins, pastes, plasters, powders, rubs, salves, shampoos, solutions, spirits, sprays, suspensions, syrups, tinctures, tonics, washes and waters) of a cardiac glycoside (condensation product of a sugar with any other radical involving the loss of the OH of the hemiacetal or hemiketal of the sugar, leaving the anomeric carbon as the link with the capacity to change the force of contraction of the heart) such as digoxin and digitoxin or pharmaceutically acceptable salt, ester, isomer or prodrug of a cardiac glycoside used or of an extract from plant species Thevetia of the family Apocynaceae or its extracts (such as 2′-o-acetylcerberoside, 2′-o-neriifolin, abonain, alpha-amyrin, arachidic-acid, aucubin, beta-amyrin, beta-amyrin-acetate, beta-sitosterol, betulin, cannogenic-acid, cannogenin, caoutchouc, cardenolides, cerberoside, eip-peruviol-acetate, hesperitin-7-glucoside, kaempferol, kokilphin, linoleic-acid, lupenyl-acetate, lupeol, lupeol-acetate, neruifolin, oleic-acid, palmitic-acid, perusitin, peruvoside, pseudoindican, quercetin, thevefoline, theveneriin, thevetin, theviridoside, theviside, triterpene, ursolic-acid) or pharmaceutically acceptable salt, ester, isomer or prodrug of an extract of the plant species Thevetia of the family Apocynaceae used for the treatment of pain (such as neuropathic, nociceptive, peripheral or central), muscle pain, muscle spasm, muscle soreness, post-exercise muscle soreness, muscle fatigue, muscle cramps, muscle stiffness, muscle strains, muscle aches, muscle twitches, myoclonus, myogenic pain, myofascial pain, muscle range of motion, muscle tension, muscle hyperalgesia or allodynia, excitability and inhabitability of motor neurons of different sizes, myofibrillar disruption, or other muscle diseases associated with muscle pain.

Such topical preparation may also contain at least one of the following ingredients: petroleum distillates, surfactants, lecithin organogel, emollients, emulsifiers (such as anionic, cationic and nonionic), irritants, counter irritants, preservatives (such as quaternary ammonium compounds, formaldehyde, halogenated phenols, sorbic acid, benzoic acid), coloring agents and suspending agents (such as sodium carboxymethyl cellulose, methylcellulose, hydroxypropylmethylcellulose, sodium alginate, polyvinylpyrrolidone, gum tragacanth and gum acacia), dispersing or wetting agents (such as naturally occurring phosphatide, e.g., lecithin), and condensation products (such as that of an alkylene oxide with fatty acids, e.g., polyoxyethylene stearate, or condensation products of ethylene oxide with long chain aliphatic alcohols, e.g, heptadecaethylene-oxycetanol, or condensation products of ethylene oxide with partial esters derived from fatty acids and a hexitol, e.g., polyoxyethylene sorbitol monooleate or condensation products of ethylene oxide with partial esters derived from fatty acids and hexitol anhydrides, e.g., polyoxyethylene sorbitan monooleate).

The composition of matter may be in combination with a nontoxic antagonist for the N-methyl-D-aspartate (NMDA) receptor (The expression “N-methyl-D-aspartate receptor” shall be understood to include all of the binding site subcategories associated with the NMDA receptor, e.g., the glycine-binding site, the phenylcyclidine (PCP)-binding site, etc., as well as the NMDA channel) such as dextromethorphan, dextrorphan or ketamine- or pharmaceutically acceptable salt, ester, isomer or prodrug of a nontoxic antagonist for the N-methyl-D-aspartate (NMDA) receptor.

The topical preparation may also contain at least one of the following ingredients: methyl salicylate, salicylic acid, aspirin (ASA), acetaminophen, Arnica montana, cajeput oil, camphor, cardamom, castor oil, cayenne (capsaicin), clove oil, cramp bark, DMSO, eucalyptus oil, ginger, guaifenesin, juniper oil, kava kava, lemon grass, lidocaine, lobelia, menthol, mint oil, non-steroidal anti-inflammatory medications, peppermint oil, skullcap, topical analgesics, topical or transdermal opioid analgesic, valerian, wintergreen oil and topica.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method of treating pain comprising topically applying to the area of pain a therapeutically effective amount of a cardiac glycoside.

2. The method of claim 1 wherein the cardiac glycoside is selected from the group consisting of digoxin, strophanthin K, digitoxin, lanatoside A, ouabain, digitoxose, gitoxin, cardenolide, oleandrin and acovenoside A.

3. The method of claim 1 wherein the cardiac glycoside is ouabain.

4. The method of claim 1 wherein the cardiac glycoside is digoxin.

5. A method of claim 1 wherein the cardiac glycoside is digitoxin.

6. The method of claim 1 wherein the cardiac glycoside is an aglycone derivative selected from the group consisting of digoxigenin, digitoxigenin, and uzarigenin.

7. The method of claim 6 wherein the aglycone derivative is digoxigenin.

8. The method of claim 1 wherein the cardiac glycoside is produced from fats or fractions thereof selected from the group consisting of fats produced from fruits of the family Palmae and seeds of the genera Garcinia, Pentadesma, Glycine, Carthamus, Olea, Brassica, Helianthus, Zea, Gossypium, Oryza, Shorea, Butyrospermum, Sesamum, Passiflora, Camelina, Limnanthes, Prunus, Triticum, Vitis, Arachis, Corylus, Persea, Madhuca, Juglans, Moringa, Macadamia, Papaver, Carica, Adenanthera, Thevetia, Trigonella, Guisotia, Pinus, Hevea, Ricinodendron, Jatropha, and Tarnarindus.

9. The method of claim 1 wherein the pain is selected from the group consisting of neuropathic, nociceptive, peripheral central, muscle pain, muscle spasm, muscle soreness, post-exercise muscle soreness, muscle fatigue, muscle cramps, muscle stiffness, muscle strains, muscle aches, muscle twitches, myoclonus, myogenic pain, myofascial pain, muscle range of motion, muscle tension, muscle hyperalgesia or allodynia, excitability and inhabitability of motor neurons of different sizes, myofibrillar disruption, and other muscle diseases associated with muscle pain.

10. The method of claim 1 wherein the cardiac glycoside is selected from the group consisting of digoxin and digitoxin or pharmaceutically acceptable salt, ester, isomer or prodrug of a cardiac glycoside used or of an extract from plant species Thevetia of the family Apocynaceae or its extracts (such as 2′-o-acetylcerberoside, 2′-o-neriifolin, abonain, alpha-amyrin, arachidic-acid, aucubin, beta-amyrin, beta-amyrin-acetate, beta-sitosterol, betulin, cannogenic-acid, cannogenin, caoutchouc, cardenolides, cerberoside, eip-peruviol-acetate, hesperitin-7-glucoside, kaempferol, kokilphin, linoleic-acid, lupenyl-acetate, lupeol, lupeol-acetate, neriifolin, oleic-acid, palmitic-acid, perusitin, peruvoside, pseudoindican, quercetin, thevefoline, theveneriin, thevetin, theviridoside, theviside, triterpene, ursolic-acid) or pharmaceutically acceptable salt, ester, isomer or prodrug of an extract of the plant species Thevetia of the family Apocynaceae.

11. The method of claim 10 wherein the topical application contains an ingredient selected from the group consisting of petroleum distillates, surfactants, lecithin organogel, emollients, emulsifiers (anionic, cationic and nonionic), irritants, counter irritants, preservatives (quaternary ammonium compounds, formaldehyde, halogenated phenols, sorbic acid, benzoic acid), coloring agents and suspending agents (sodium carboxymethyl cellulose, methylcellulose, hydroxypropylmethylcellulose, sodium alginate, polyvinylpyrrolidone, gum tragacanth and gum acacia), dispersing or wetting agents (naturally occurring phosphatide, lecithin), condensation products (of an alkylene oxide with fatty acids, polyoxyethylene stearate, or condensation products of ethylene oxide with long chain aliphatic alcohols, heptadecaethylene-oxycetanol, condensation products of ethylene oxide with partial esters derived from fatty acids, and a hexitol, polyoxyethylene sorbitol monooleate or condensation products of ethylene oxide with partial esters derived from fatty acids, hexitol anhydrides, and polyoxyethylene sorbitan monooleate).

12. The method of claim 10 wherein the topical application is in combination with a nontoxic antagonist for the N-methyl-D-aspartate (NMDA) receptor.

13. The method of claim 1 wherein the topical application includes an ingredient selected from the group consisting of methyl salicylate, salicylic acid, aspirin (ASA), acetaminophen, Arnica montana, cajeput oil, camphor, cardamom, castor oil, cayenne (capsaicin), clove oil, cramp bark, DMSO, eucalyptus oil, ginger, guaifenesin, juniper oil, kava kava, lemon grass, lidocaine, lobelia, menthol, mint oil, non-steroidal anti-inflammatory medications, peppermint oil, skullcap, topical analgesics, topical or transdermal opioid analgesic, valerian, wintergreen oil and topica.

14. A composition of matter comprising a topical application being of a form selected from the group consisting of aqueous acids, aromatic waters, balms, cataplasms, poultices, collodions, creams, dressings, elixirs, emulsions, extracts, fluidextracts, foams, gels, glycerins, honeys, irrigation solutions, jellies, juices, liniments, liposomes, lotions, magmas, microemulsion, milks, mixtures, mucilages, oils, ointments (oleaginous, anhydrous, water in oil, oil in water and water soluble bases), oleovitamins, pastes, plasters, powders, rubs, salves, shampoos, solutions, spirits, sprays, suspensions, syrups, tinctures, tonics, washes and waters), and the topical application being a cardiac glycoside selected from the group consisting of digoxin and digitoxin or pharmaceutically acceptable salt, ester, isomer and prodrug of a cardiac blycoside.

15. The composition of matter as claimed in claim 14 wherein the topical preparation also contains at least one ingredient selected from the group consisting of petroleum distillates, surfactants, lecithin organogel, emollients, emulsifiers (anionic, cationic and nonionic), irritants, counter irritants, preservatives (quaternary ammonium compounds, formalyde, halogenated phenols, sorbic acid, benzoic acid), coloring agents and suspending agents (sodium carboxymethyl cellulose, methylcellulose, hydroxypropylmethylcellulose, sodium alginate, polyvinylpyrrolidone, gum tragacanth and gum acacia), dispersing or wetting agents (naturally occurring phosphatide, lecithin), and condensation products.

16. The composition of matter as claimed in claim 14 in combination with a nontoxic antagonist for the N-methyl-D-aspartate (NMDA) receptor.

17. The composition of matter as claimed in claim 14 wherein the topical preparation also contains at least one ingredient selected from the group consisting of methyl salicylate, salicylic acid, aspirin (ASA), acetaminophen, Arnica montana, cajeput oil, camphor, cardamom, castor oil, cayenne (capsaicin), clove oil, cramp bark, DMSO, eucalyptus oil, ginger, guaifenesin, juniper oil, kava kava, lemon grass, lidocaine, lobelia, menthol, mint oil, non-steroidal anti-inflammatory medications, peppermint oil, skullcap, topical analgesics, topical or transdermal opioid analgesic, valerian, wintergreen oil and topical anti-inflammatory agents.
